(19) United States
(12) Patent Application Publication
(10) Pub. No.: US 2011/0137423 A1
(43) Pub. Date: Jun. 9, 2011

(54) Mechanical joint imitating creatures' joints

(76) Inventor: Hsiang Ouyang, San Gabriel, CA (US)

(21) Appl. No.: 12/591,910

(22) Filed: Dec. 4, 2009

(51) Int. Cl. A61F 2/30

(52) U.S. Cl. 623/18.11

(57) ABSTRACT

A mechanical joint system comprises at least a pair of segments swivelable relative to each other. The swivel of the segments are actuated by ligament(s) and the ligament(s) are actuated by either or the combination of “a two-direction control by force transmitting device,” “a right-hand thread and left-hand thread co-existence mechanism” and “an extendible and retractable mechanism.” The three actuation ways simulate relaxation and tension of creatures' ligaments and muscles by using threaded cylinders and nuts, and can replace conventional motor units in order to reduce the size, weight and cost of a robot.

### BACKGROUND OF THE INVENTION

1. Field of the Invention

The present invention relates to a mechanical system imitating creatures' joint movements to achieve daily and general functions. More particularly, the present invention relates to a mechanical system using a threaded cylinder combining the right-hand thread and left-hand thread co-existence mechanism, two-direction control by force transmitting device and a hybrid threaded cylinder bouncing structure. It can considerably decrease the number of the components used to control the joint movements, and make the circuit design less complicated than ever, so that the size and the cost of the mechanical system can be reduced.

2. Description of the Prior Art

Automatically controlled mechanical limbs are widely used in industrial manufacturing processes for mass production, but have not been well adapted to the fields of prosthesis, artificial joint and robot for, for example, harvesting fruits. That is because these automatically controlled mechanical limbs look different from a human arm. Current mechanical limbs for industrial use actually look like a fixture, rather than a human hand with fingers and joints. Industrial mechanical limbs, generally called “robots,” are mainly for achieving numerous single and precise movements, such as picking a workpiece up from a specific location and putting a workpiece down in a specific location. Conventional mechanical limbs are configured differently in accordance with the form and shape of the workpiece desired to be grasped. They are suitable for manufacturing standardized industrial products only.

In contrast, human hands or animal's paws have a variety of functions. They are significantly different from most of the mechanical limbs which are used to do standardized work. A human hand can deal with a variety of conditions which may not need the precision of a robot. Restricted by control and driving units, such as motors, a robot can hardly rotate about several axes and at different angles nimbly like a human hand.

Therefore, there is a need to invent a mechanical limb by imitating a human hand to meet daily ordinary requirements and also reduce the size and simplify the control circuits.

The nimbleness of a robot depends on its “degree of freedom.” When an article can swivel about or move relative to a specific axis, the article has one degree of freedom. For example, the knuckle near the tip of an index finger can swivel in one direction, that is, the finger segment can straighten or bend about one axis only, so this knuckle has one degree of freedom only. The knuckles connecting the fingers and the palm have three degrees of freedom because these knuckles allow the fingers to swivel forward, swing to left and right, and draw a circle with the fingertip. In conventional technology, the degrees of freedom are proportional to the number of motors. It takes at least one driving device, such as a motor, and the device to control the driving device, such as a servo unit, to drive and control any of the joints of a conventional mechanical limb.

A human hand has 24 degrees of freedom (four for the thumb and five for each of the other fingers). A mechanical limb can be as nimble as a human hand if it also has 24 degrees of freedom. In conventional technology, each degree of freedom is achieved by one motor to drive the mechanism to swivel about one axis. Therefore, a mechanical limb would need 24 motors to have the same degrees of freedom as a human hand and each of the motors must be sufficiently small to keep the size of the robot appropriate. As a small motor cannot provide enough torque, such a mechanical limb may not be able to grasp anything. If motors with large torque are used, the mechanical limb may become too large and too heavy to be practical.

Conventional technology takes advantage of the high precision of a step motor and its digit controllability to manipulate the movement of the knuckles of a robot. To reduce complexity, weight and cost and improve practicability, a conventional robot usually has two to five joints only. An extraordinarily new technical concept is required to make a robot able to imitate the movements of human fingers and grasp articles in practice. The present invention escapes from the conventional concept which focuses on developing complicated and precise control with servo motors. The present invention uses the threaded cylinder and nut structure to replace part of the conventional motor units in order to reduce the size, weight and cost. The present invention further uses ligament-like structure to pull and control the movement of the knuckle. It is closer to the real creatures' joints or knuckles.

### SUMMARY OF THE INVENTION

An object of the present invention is to reduce the number of components used in a robot so as to reduce the size and weight of the robot, and the control circuits. Because it does not use a motor to drive every joint, the servo for controlling the motor and the motor drivers are also not necessary.

Another object of the present invention is to provide an inventive robot mechanism which reduces the use of step motors or the servo motor units and motor drivers and therefore reduces the cost.

A further object of the present invention is to save power for maintaining the holding torque of the step motor in the holding mode with no or less step motors used, but the swivel state of a joint can still be held.

The present invention can also reduce heat emission during operation. Because a step motor in a holding mode needs a large current to generate large torque and this generates heat, in some situations, the motor further needs a heat dissipation mechanism to prevent it from burning down. The present invention uses no or less step motors, so the heat emission is reduced.

A further object of the present invention is to provide a robot suitable for working in a humid condition or in water. The joints of the robot according to the present invention can be driven by hydraulic or pneumatic power. Using hydraulic or pneumatic power is cheaper and more suitable for tasks in humid conditions than using electromagnetic power.

The present invention provides a mechanical joint system, comprising: a joint unit including a first segment, a second segment, a jointing portion connecting the first segment and the second segment and allowing the first segment and the second segment to swivel about the jointing portion, a first ligament with one end connected to a first connection of the first segment and a second ligament with one end connected to a second connection of the first segment, a threaded cylinder having a right-hand thread section or a left-hand thread section, a nut fitted on the thread section and constrained by a guide from rotating with the thread section, and a force-transmitting device; wherein: the first ligament is guided by the force-transmitting device and connected to the nut at the other end, the second ligament is connected to the nut at the other end; and the force transmitting device makes the section of the first ligament between the end connected to the first segment and the force-transmitting device move in the direction different from the section of the first ligament between the other end connected to the nut and the force-transmitting device.

The subject invention also provides a mechanical joint system, comprising: a joint unit including a first segment, a second segment, a jointing portion connecting the first segment and the second segment and allowing the first segment and the second segment to swivel about the jointing portion, a right-hand thread and left-hand thread co-existence mechanism, a first ligament with one end connected to a first connection of the first segment and a second ligament with one end connected to a second connection of the first segment; wherein: the right-hand thread and left-hand thread co-existence mechanism comprises a threaded cylinder having a right-hand thread section and a left-hand thread section in pair, a first nut is fitted on the right-hand thread section, and a second nut is fitted on the left-hand thread section; and the first nut and the second nut are prevented by a guide from rotating with the right-hand and left-hand thread sections; and the other end of the first ligament is connected to the first nut and the other end of the second ligament is connected to the second nut.

The present invention further provides a mechanical joint system, comprising:
a joint unit including a first segment, a second segment, a jointing portion connecting the first segment and the second segment and allowing the first segment and the second segment to swivel about the jointing portion, an extendible and retractable cylinder mechanism, a first ligament with one end connected to a first connection of the first segment and a second ligament with one end connected to a second connection of the first segment; wherein: the extendible and retractable cylinder mechanism comprises a rail, a first cylinder, a second cylinder, a first block fitted to the first cylinder, a second block fitted to the second cylinder, and a driving device; and the first and second cylinders slide along the rail in opposite directions when the driving device is initiated to drive the first and second cylinders; the first block and the second block are prevented from rotating with the first and second cylinders; and the other end of the first ligament is connected to the first block and the other end of the second ligament is connected to the second block.

### BRIEF DESCRIPTION OF THE DRAWINGS

FIG. 1 shows two pairs of ordinary threaded cylinders and nuts threaded in opposite directions.

FIG. 2A is the first embodiment of the present invention.

FIG. 2B is the second embodiment of the present invention.

FIG. 3 is the third embodiment of the present invention.

FIG. 4 illustrates a different type of threaded cylinders and a different type of nuts used in the fourth embodiment of the present invention.

FIG. 5 shows a set of threaded cylinders and nuts according to the present invention is arranged on a base of a robot.

FIG. 6 illustrates a guide for constraining a nut fitted on a threaded cylinder of the present invention.

FIG. 7A illustrates an assembly of the joint unit, the rigid ligament and the threaded cylinders according to the present invention.

FIG. 7B is the nut 60 of the mechanical finger assembly shown in FIG. 7A.

FIG. 7C is the nut 50 of the mechanical finger assembly shown in FIG. 7A.

FIGS. 8A-8C show the structures of the components used in the mechanical finger assembly shown in FIG. 7A.

FIG. 9A is another view of the mechanical finger assembly in FIG. 7A.

FIGS. 9B-9C are another views of the nuts 50 and 60 in FIGS. 7B and 7C.

FIG. 10A is another embodiment of the mechanical finger assembly according to the present invention.

FIG. 10B is the nut 50 used in the mechanical finger assembly in FIG. 10A.

FIG. 11 is an embodiment of the “extendible and retractable threaded cylinder” according to the present invention.

FIG. 12 is an example of the components of the “extendible and retractable threaded cylinder” according to the present invention.

FIG. 13 is another example of the components of the “extendible and retractable threaded cylinder” according to the present invention.

### DETAILED DESCRIPTION

The following is a description of how the present invention uses two-direction control by force transmitting device and a right-hand thread and left-hand thread co-existence mechanism to control the swivel of a mechanical joint with reference to the drawings. An extendible and retractable threaded cylinder mechanism can be further incorporated to imitate an actual creature's joint.

FIG. 1 shows threaded cylinders 3 and 4 which are threaded with right-hand and left-hand threads respectively. Nuts 5 and 6 are fitted to the threaded cylinders 3 and 4 respectively. When the threaded cylinders 3 and 4 spin in the clockwise direction, the nuts 5 and 6 move along the axes of the threaded cylinders 3 and 4 in the opposite direction.

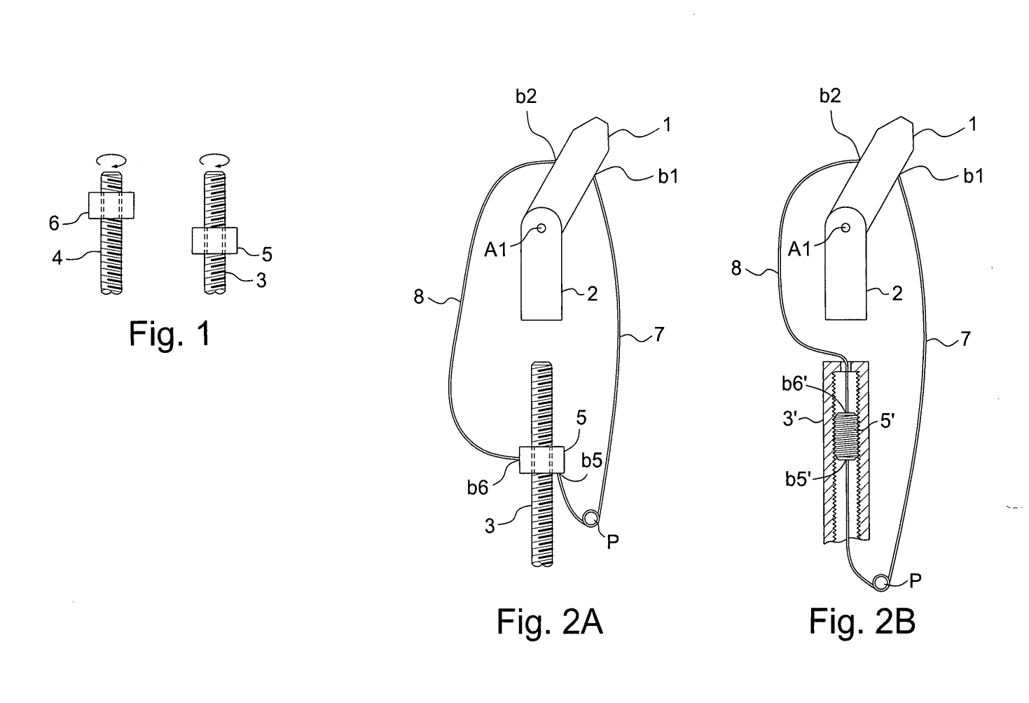

FIG. 2A and FIG. 2B illustrate embodiments of the two-direction control by force transmitting device. The two embodiments use a spinning cylinder threaded in one (right-hand or left-hand) direction, a nut fitted thereto and a pulley or a set of pulleys which guides one of the two ligaments to transmit the force and change force direction, thereby achieving the effect of swiveling the segment of a mechanical joint. The details are as follows:

FIG. 2A is a first embodiment of the two-direction control by force transmitting device. A threaded cylinder 3 threaded with a right-hand thread or a left-hand thread is provided. A nut 5 (or a block 5 which has a threaded structure for matching with the thread of the threaded cylinder 3 and moves relative to the threaded cylinder 3 when the threaded cylinder 3 spins) is engaged with the threaded cylinder 3. When the threaded cylinder 3 spins, the nut (or block) 5 is prevented from rotating along with the threaded cylinder 3 and therefore moves along the threaded cylinder 3. In FIG. 2A, element 1 and element 2 which are connected by a jointing portion A1 are the first segment 1 and the second segment 2 of a mechanical finger. The first segment 1 is able to be swiveled relative to the second segment 2, and a joint unit is constructed thereby. A first ligament 7 is connected to a first connection b1 of the first segment 1 at one end and a second ligament 8 is connected to a second connection b2 of the first segment 1 at one end. The first ligament 7 is guided by a force transmitting device P, which can be a pulley or a set of pulleys, and then connected to a connection b5 of the nut 5 at the other end. The second ligament 8 is connected to another connection b6 of the nut 5 with the other end. When the threaded cylinder 3 spins, the nut 5 moves along the threaded cylinder 3 because it is constrained from rotating along with the threaded cylinder 3. The set of pulleys P guides the first ligament 7 to transmit force in a direction opposite to the force direction of the second ligament 8. When the nut 5 moves upwardly, the first ligament 7 is tensed gradually and the second ligament 8 is relaxed gradually. When the nut 5 moves downwardly, the first ligament 7 is relaxed gradually and the second ligament 8 is tensed gradually. This generates a relative movement of “push and pull, ” so that the first segment 1 can bend or straighten relative to the second segment 2 just like a creature's joint controlled by muscles.

FIG. 2B is a second embodiment of the two-direction control by force transmitting device. A threaded cylinder 3′ has an inner passage with a single-direction (right or left-hand) female thread formed therein. A block 5′ with a threaded structure for matching with the female thread(s) of the threaded cylinder 3′ is fitted in the inner passage of the threaded cylinder 3′. When the threaded cylinder 3′ spins, the block 5′ is prevented from rotating along with the threaded cylinder 3′ and therefore moves along the threaded cylinder 3′. In FIG. 2B, the first segment 1 and the second segment 2 which are connected by the jointing portion A1 are able to be swiveled relative to each other, and a joint unit is constructed thereby. The first ligament 7 is connected to the first connection b1 of the first segment 1 at one end and the second ligament 8 is connected to the second connection b2 of the first segment 1 at one end. The first ligament 7 is guided by the force transmitting device P, which can be a pulley or a set of pulleys, and then connected to a connection b5′ of the block 5′ at the other end. The second ligament 8 is connected to another connection b6′ of the block 5′ at the other end. When the threaded cylinder 3′ is spinning, the set of pulleys P guides the first ligament 7 to transmit force in a direction opposite to the force direction of the second ligament 8. When the block 5′ moves upward, the first ligament 7 is tensed gradually and the second ligament 8 is relaxed gradually. When the block 5′ moves downward, the first ligament 7 is relaxed gradually and the second ligament 8 is tensed gradually. This generates a dual-side relative movement of “push and pull,” so that the first segment 1 can bend or straighten relative to the second segment 2 just like a creature's joint controlled by muscles.

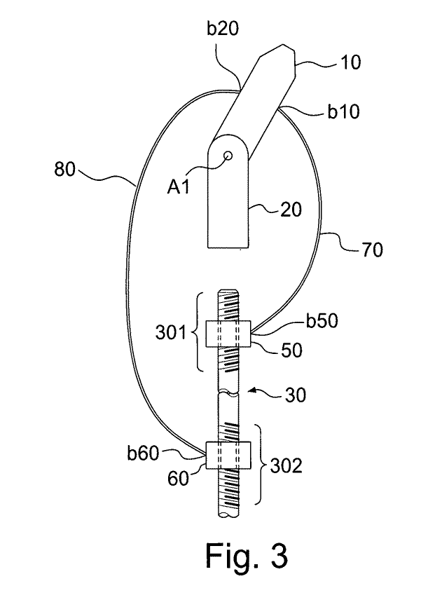

Another embodiment of the present invention takes advantage of a right-hand thread and left-hand thread co-existence mechanism to control a mechanical joint. FIG. 3 shows a cylinder 30 with a right-hand thread section 301 and a left-hand thread section 302. When the threaded cylinder 30 having the right-hand and left-hand thread sections 301 and 302 spins, a first nut (or block) 50 and a second nut (or block) 60 which have threaded structure for being engaged with the right-hand and left-hand thread sections 301 and 302 respectively move in opposite directions. A mechanical finger has a first segment 10 and a second segment 20, both of which are connected by a jointing portion A1 and able to be swiveled relative to each other; a joint unit is constructed thereby. The first ligament 70 is connected to the first connection b10 of the first segment 10 at one end and the second ligament 80 is connected to the second connection b20 of the first segment 10 at one end. The other end of the first ligament 70 is connected to a connection b50 of the nut 50 fitted to the right-hand thread section 301 of the threaded cylinder 30. The other end of the second ligament 80 is connected to another connection b60 of the nut 60 fitted to the left-hand thread section 302 of the threaded cylinder 30. When the threaded cylinder 30 spins, the nuts 50 and 60 are prevented from rotating along with the threaded cylinder 30 and move toward opposite directions along the threaded cylinder 30. When the nuts 50 and 60 move in each other, the first ligament 70 is tensed gradually and the second ligament 80 is relaxed gradually. When the nuts 50 and 60 move away from each other, the first ligament 70 is relaxed gradually and the second ligament 80 is tensed gradually. This generates a dual-side relative movement of “push and pull,” so that the first segment 1 can bend or straighten relative to the second segment 2 just like a creature's joint controlled by muscles.

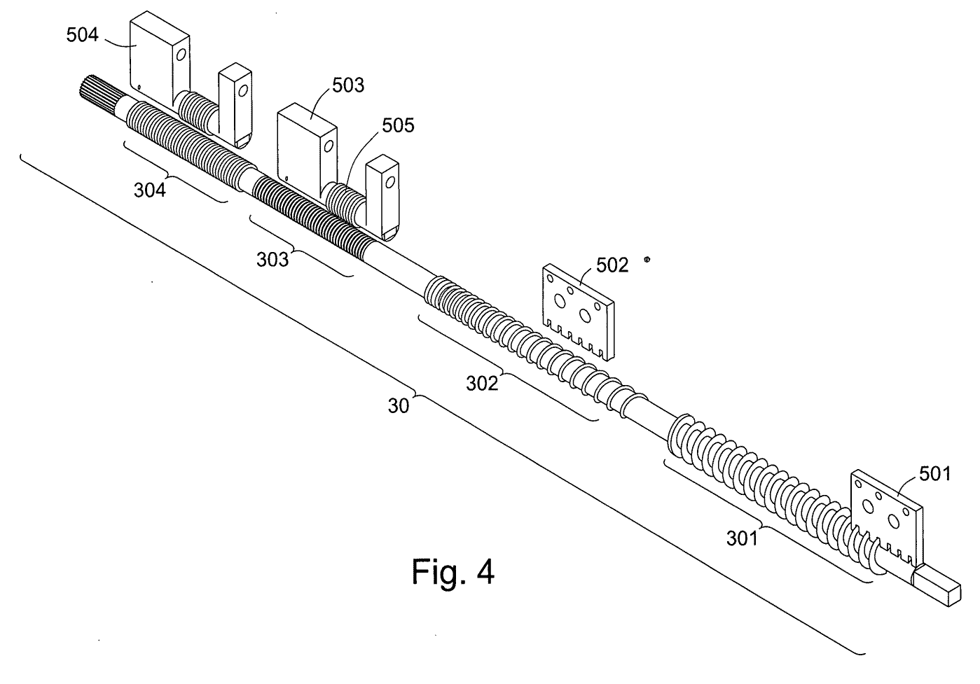

FIG. 4 is another embodiment of the right-hand thread and left-hand thread co-existence mechanism. The threaded cylinder 30 has a right-hand thread section 301 and a left-hand thread section 302 and another pair of a right-hand thread section 303 and a left-hand thread section 304. The nuts mentioned in the previous embodiment are changed to a block 501 and a block 502 having threading teeth; the pitches of the threading teeth are the same as the pitches of the corresponding thread sections of the threaded cylinder 30. The plurality of holes formed on the blocks 501 and 502 is to provide changeable locations of the connection to the ligament. The nuts mentioned in the previous embodiment can also be changed to blocks 503 and 504 having a threaded cylinder for matching with the right-hand and left-hand thread sections 303 and 304 respectively. The blocks 501, 502, 503, 504 are constrained by a guide (not shown in FIG. 4) so that they will not rotate along with the threaded cylinder 30. Since the threaded cylinder 30 in FIG. 4 has two pairs of right-hand and left-hand thread cylinders 301, 302, 303, 304, it is able to drive two mechanical joint units to swivel when spinning. To drive more mechanical joint units, three or more pairs of right-hand and left-hand thread sections can be provided on a threaded cylinder. These right-hand and left-hand thread sections and the nuts or blocks fitted thereon can be provided with different pitches or different types of threading structure, so that the joint units' maximum swivel angles may vary and the joints may swivel at different angular velocities.

The threaded cylinder 30 with a male thread(s) in FIG. 4 can be changed to a cylinder with several right-hand and left-hand thread sections formed of female threads like the threaded cylinder 3′ shown in FIG. 2B, and the blocks having male threads for matching with these female right-hand and left-hand thread sections are provided. In addition to replacing the threaded cylinder of male threads by a cylinder of female threads, the threaded cylinder can be made to have both of female and male threads. A right-hand and a left-hand thread sections in a pair can have different types of threads in order to generate differential speed. Several pairs of right-hand and left-hand thread sections with different types of threads can generate different speeds and move the nuts or blocks in different directions. This is an application of the worm and gear concept. The friction between the engaging surfaces of the threaded cylinder and the nut can be reduced by providing a ball-bearing as the interface.

FIG. 5 shows that two threaded cylinders with right-hand and left-hand thread sections formed thereon and one threaded cylinder with a single-direction thread are fitted in a hollow base 90 with their ends inserted into the holes formed on the top and bottom plates of the base 90. The threaded cylinders are able to spin after fitted. The base 90 is equivalent to the bone or shell of a creature. Inlaying a set of threaded cylinders in a base 90 makes it unnecessary to provide other separate supports for individual threaded cylinders. When these threaded cylinders spin at the same time, the nuts engaged with the threaded cylinders drive several mechanical joint units to swivel or translate. The base 90 may have more holes for holding threaded cylinders than actually needed. In practice, one or more threaded cylinders are selected according to the actual need and fitted to the holes of the base 90 to drive one or more mechanical joint units to swivel or translate, so as to achieve the required motion, such as clip and grasp.

The two-direction control by force transmitting device and a right-hand thread and left-hand thread co-existence mechanism associated with ligaments to tense and/or relax a segment can save circuits and driving devices (such as motors). The more pairs of right-hand and left-hand thread sections exist on a threaded cylinder, the more driving devices and circuits are saved. The power for driving the threaded cylinders of the present invention can be magnetic force, hydraulic or pneumatic power, thermal energy, creatures' forces, villus movements, wind power, solar energy or any other external forces.

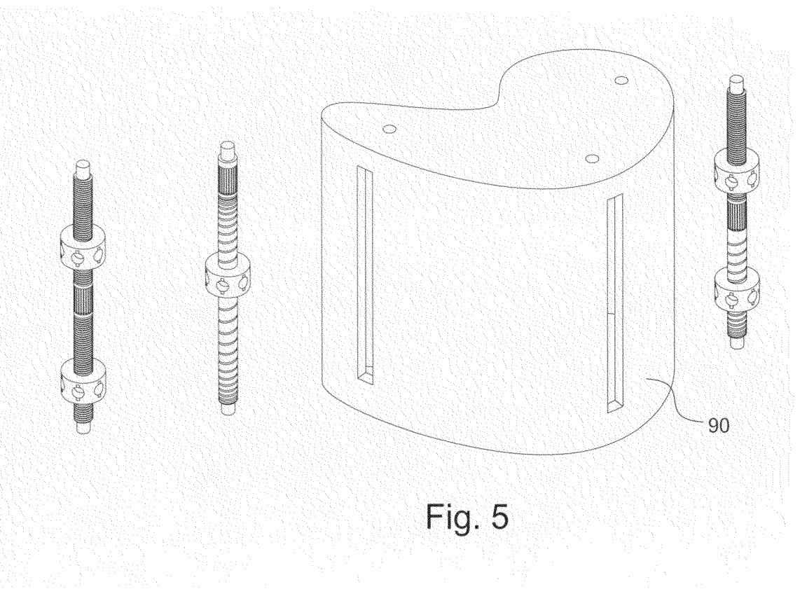

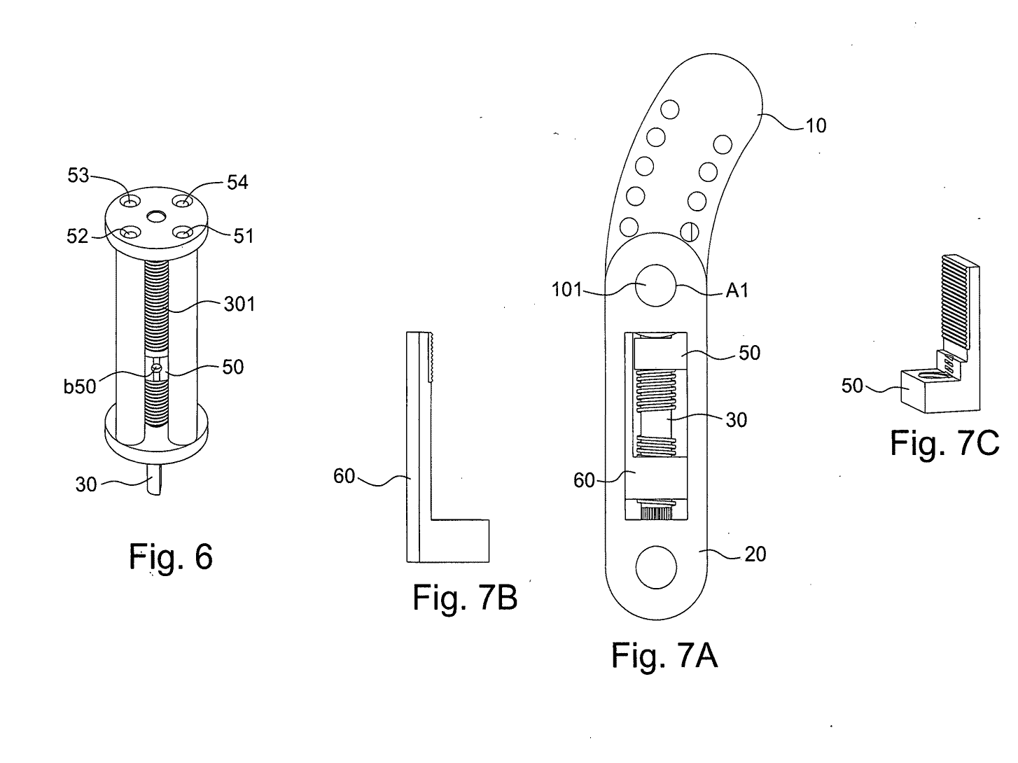

FIG. 6 shows four columns 51, 52, 53, 54 surrounding the nut 50 to prevent it from rotating. This restriction on a nut's rotation can be achieved by using a nut with hexagonal or other non-circular contour, and the points or surfaces that the four columns 51, 52, 53, 54 contact the outer contour of the nut 50 form a guide to prevent the nut 50 from rotating along with the threaded cylinder. Alternatively, the rotation of the nut 50 can be constrained by a stick extending from the location of the connection b50 on the nut 50 to the gap between the columns 51 and 52. The nut 50 is therefore moved upward or downward when the threaded cylinder 30 spins. The guide can be any other forms as long as the rotation of the nut can be constrained.

The ligament for connecting the segment and the nut can be made of soft and/or flexible material. For a soft ligament, flexible material is preferred to provide a gentle grasp suitable for grasping an egg or fruit. The locations of the connections b10 and b20 are adjustable. By adjusting the locations of the connections of the ligaments, the torque with which the ligaments make the segment swivel is increased or reduced; therefore, the maximum rotation angle of the segment at the same translation distance of the nut can be varied. Several maximum swivel angles of a joint can thus be predetermined.

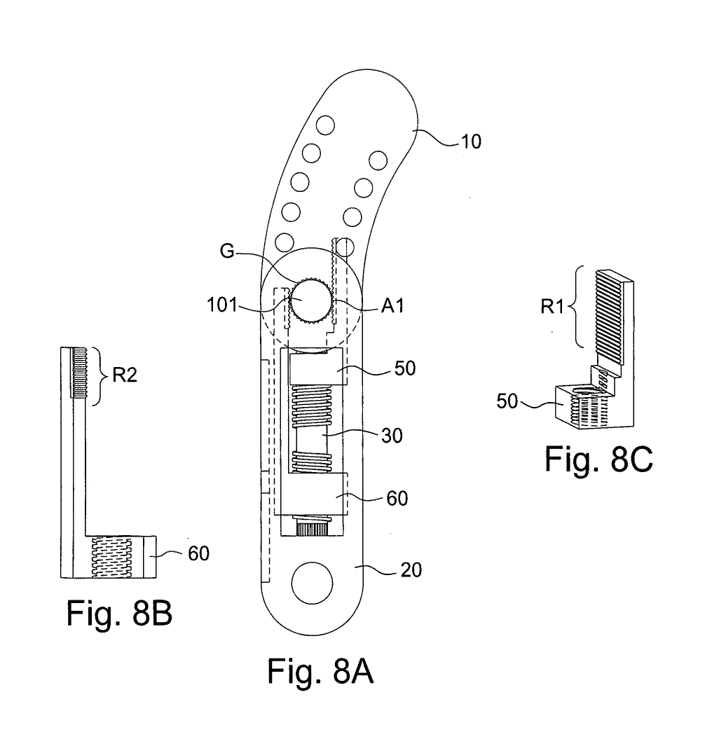

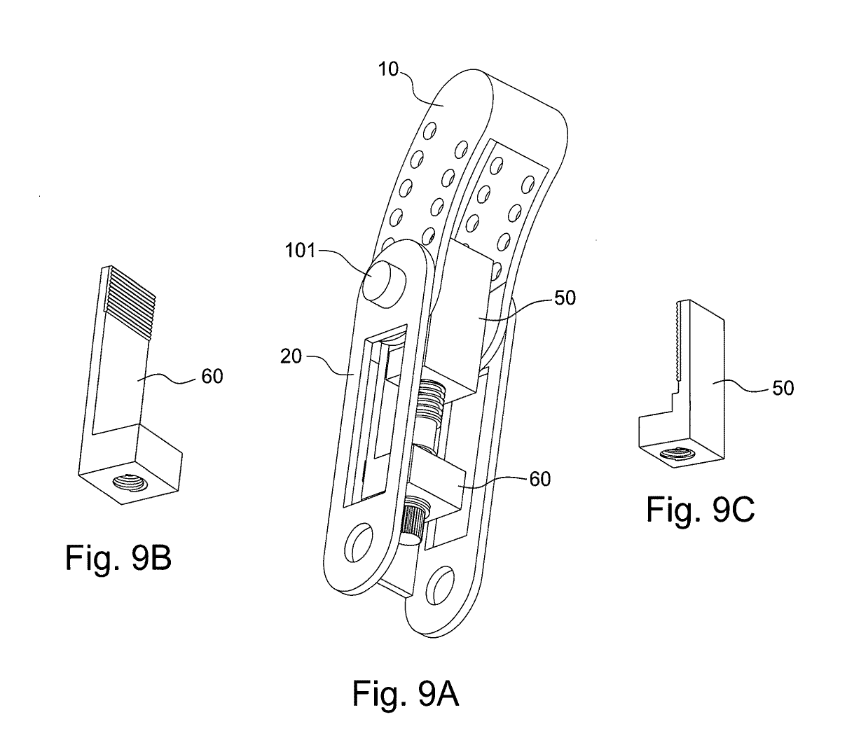

FIGS. 7A-10B show how the above mentioned two-direction control by force transmitting device and a right-hand thread and left-hand thread co-existence mechanism are embedded in a mechanical finger. Instead of soft ligaments intuitively used as illustrated in the previous embodiments, FIGS. 7A-10B show the practical designs of using rigid ligaments. A swivel axle 101 extends from the jointing portion A1 of the first segment 10 and passes through the swivel hole on the second segment 20 (FIG. 7A), and a gear G (FIG. 8A) is formed as the central portion of the axle 101 located at the interior hollow portion of the first segment 10. The nuts 50 and 60 are engaged with the right-hand and left-hand thread sections of the threaded cylinder 30 respectively and further have racks R1 and R2 (with teeth) extending therefrom respectively. The teeth of the racks R1 and R2 are engaged with the gear G to form a rigid ligament. When the nuts 50 and 60 move along the threaded cylinder 30 as a result of the spinning of the threaded cylinder 30, the racks R1 and R2 drive the gear G of the axle 101, thereby swiveling the first segment 10 relative to the second segment 20.

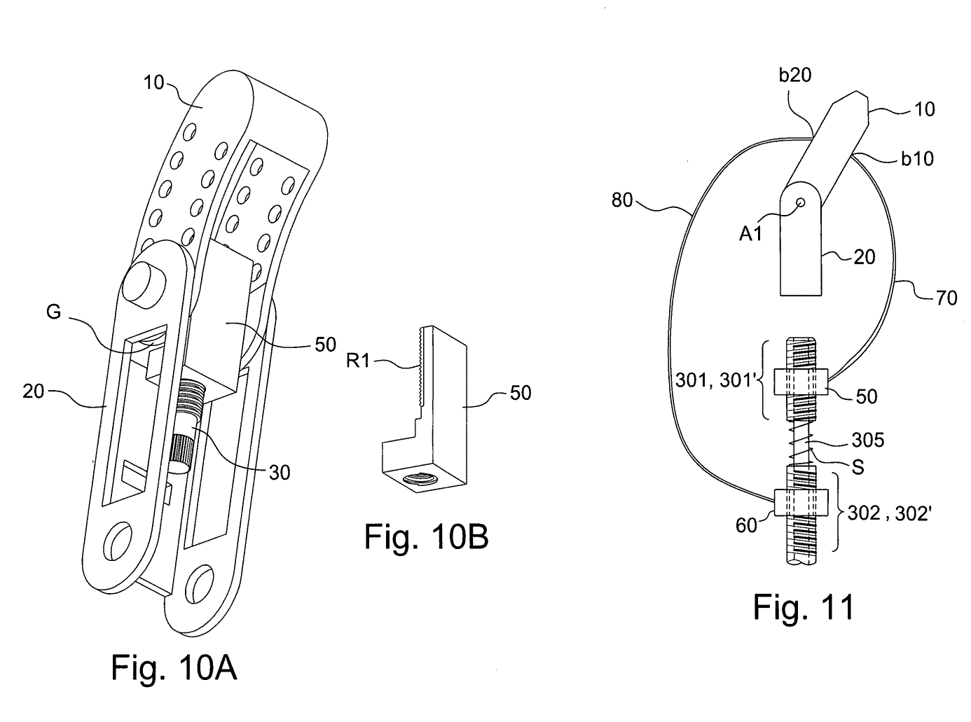

FIG. 10A is another embodiment with the nut 60 and the rack R2 shown in FIG. 8A removed. With the nut 50 engaged with a single-direction thread section of the threaded cylinder 30 and the rack R1 engaged with the gear G, the two-direction control by force transmitting device is achieved. That is because the rigidity of the rack R1 and the gear G is able to transmit not only a clockwise torque to the first segment 10 when the gear G rotates in the clockwise direction, but also a counterclockwise torque to the first segment 10 when the gear G rotates in the counterclockwise direction. The function of a pulley for transmitting a force carried by a soft ligament to a different direction is therefore achieved.

A soft ligament can be a steel cable, chain or track with sufficient strength or made of composite materials, such as nylon and fibers.

One right-hand thread and left-hand thread co-existence mechanism can generate one degree of freedom for rotation. If more than one is used, by adjusting the locations of the ligaments' connections to the first segment, the joint unit can perform a 3-dimensional motion to simulate a 360-degree swivel of a finger. The present invention can apply to a solar energy system to simulate the motion of a sunflower to swivel the sunlight receiving apparatus to follow the sun.

The above mentioned structure can further be combined with an extendible and retractable mechanism to become an extendible and retractable threaded cylinder, in order to instantly swivel a joint. In other words, a hybrid threaded cylinder and slider-slot structure is adopted. In addition to the nuts' movements in different directions as a result of different types of threads, the extendible and retractable threaded cylinder performs an instant movement at two or more speeds, so the mechanical joint performs a fillip or a bounce motion which is like the performance of an animal motion requiring explosive force.

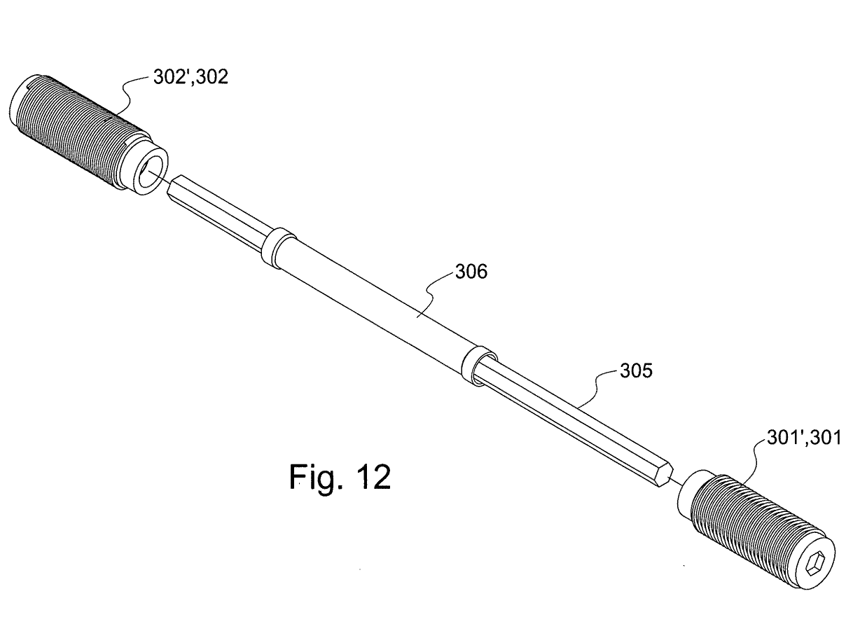

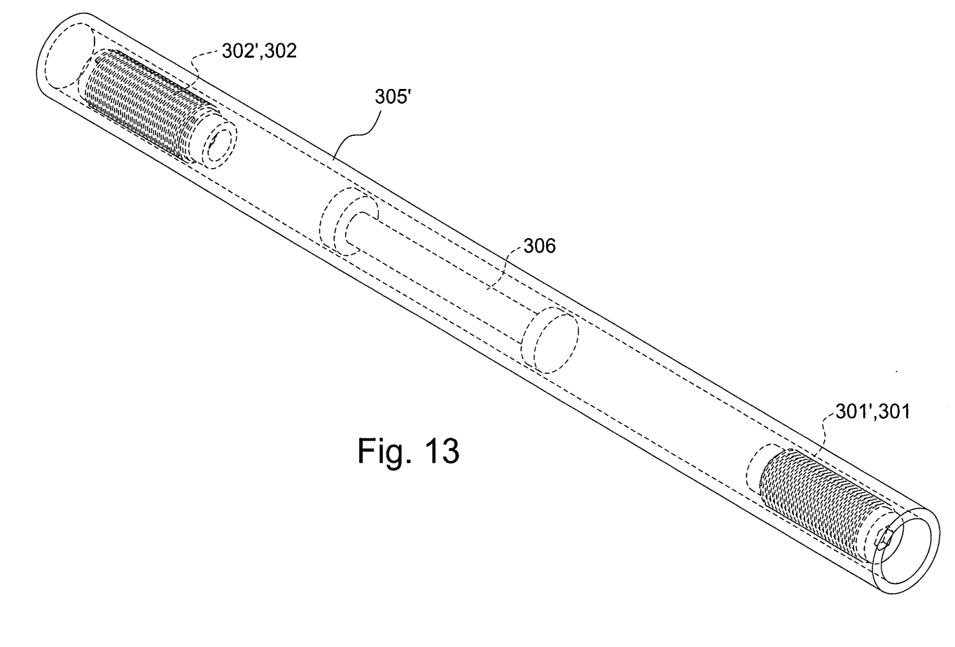

FIG. 11 illustrates an extendible and retractable threaded cylinder able to drive a joint unit to swivel. The threaded cylinder illustrated in the previous embodiments is divided into a hollow first cylinder 301′ and a hollow second cylinder 302′, of which one is the right-hand thread section 301 and the other is the left-hand thread section 302. A shaft 305 is inserted into the first and second cylinders 301′ and 302′, so that the first cylinder 301′ and the second cylinder 302′ can slide along the shaft 305. A high-tension spring S can be used to connect the first cylinder 301′ and the second cylinder 302′ and drive them to slide along the shaft 305 relative to each other. If there is no need to slide the cylinders 301′, 302′ along the shaft 305, the relative positions of the first cylinder 301′ and the second cylinder 302′ are fixed with a locking mechanism (not shown); the opening and closing of the locking mechanism is controlled by, for example, a relay or a solenoid. When there is a need to slide the first cylinder 301′ and the second cylinder 302′ along the shaft 305, an electrical signal is sent to the relay to release the locking mechanism, and then the cylinders 301′ and 302′ with the right-hand and left-hand thread sections 301 and 302 and the nuts 50 and 60 thereon are driven by the spring force to instantly bounce upward and downward; the ligament 80 is suddenly tensed and the ligament 70 is suddenly relaxed, thereby swiveling the joint unit very fast. The spring force for driving the right-hand and left-hand thread sections 301 and 302 can be replaced with pneumatic power or other power. For example, the right-hand and left-hand thread sections 301 and 302 can be connected to a pneumatic cylinder or a hydraulic cylinder or driven by a magnetic force. The nuts' movements relative to the right-hand and left-hand thread sections 301 and 302 and the extending or retracting movement of the threaded cylinders 301′ and 302′ can be triggered at the same time. The shaft 305 can be configured to guide the first cylinder 301′ and the second cylinder 302′ to slide in a linear or curved route. Further, the “extendible and retractable threaded cylinder” compensates the movement requiring high-torque for fast moving speed. FIG. 12 is an example of the separate right-hand and left-hand thread sections 301 and 302 and the shaft 305. A barrel 306 tubes the shaft 305 and is located between the first cylinder 301′ and the second cylinder 302′ to keep a certain distance between the two threaded cylinders 301′ and 302′. The shaft 305 used for guiding the extendible and retractable threaded cylinders to slide can be replaced with any other suitable guiding structures. For example, the threaded cylinders 301′, 302′ can be fitted in a “chamber” 305′ in FIG. 13; the inner contour of the chamber 305′ acts as a rail to guide the threaded cylinders 301′ and 302′ to slide. Alternatively, the threaded cylinders 301′ and 302′ can be provided with a structure or a device able to be fitted to and slide on a rail available in the market and not blocking the spinning of the threaded cylinders 301′ and 302′.

The present invention successfully simplifies the complicated structure of a conventional mechanical joint and reduces the use of expensive control devices. The size of the mechanical joint and finger can be considerably reduced. According to the present invention, the diameter of a joint unit controlled by a computer can be reduced to 0.5 mm. Because the mechanisms can be miniaturized, the modulized mechanical joint can be easily put into the mechanical limb to be controlled.

The present invention can reduce the number of motors used. The disadvantage of motors is that when the size of a motor is reduced, its holding torque will be reduced too. That is why a small motor needs an additional gear box to increase the holding torque. The present invention uses very few motors, reduces the size and cost of a mechanical limb and keeps the holding torque at a sufficiently high level.

The joints of the mechanical limb according to the present invention can be driven by hydraulic and/or pneumatic power, or by electric or magnetic power. A water-proof mechanical limb driven by hydraulic power is cost-efficient, so it is more suitable for a humid environment or even in water than one driven by electromagnetic motors.

The embodiments described above are for illustration only. The variations and modifications may be made according to the spirit of the present invention or the equivalent scope. They should be considered as falling within the coverage of description of the present invention.

We claim:

1. A mechanical joint system, comprising:
a joint unit including a first segment, a second segment, a jointing portion (A1) connecting the first segment and the second segment and allowing the first segment and the second segment to swivel about the jointing portion (A1), a first ligament with one end connected to a first connection (b1) of the first segment and a second ligament with one end connected to a second connection (b2) of the first segment, a threaded cylinder having a right-hand thread section or a left-hand thread section, a nut fitted on the thread section and constrained by a guide from rotating with the thread section, and a force-transmitting device; wherein:
the first ligament is guided by the force-transmitting device and connected to the nut at the other end, the second ligament is connected to the nut at the other end; and the force transmitting device makes the section of the first ligament between the end connected to the first segment and the force-transmitting device move in the direction different from the section of the first ligament between the other end connected to the nut and the force-transmitting device.

2. A mechanical joint system according to claim 1, wherein the force-transmitting device is a pulley.

3. A mechanical joint system, comprising:
a joint unit including a first segment, a second segment, a jointing portion (A1) connecting the first segment and the second segment and allowing the first segment and the second segment to swivel about the jointing portion (A1), a right-hand thread and left-hand thread co-existence mechanism, a first ligament with one end connected to a first connection (b10) of the first segment and a second ligament with one end connected to a second connection (b20) of the first segment; wherein:
the right-hand thread and left-hand thread co-existence mechanism comprises a threaded cylinder having a right-hand thread section and a left-hand thread section in pair, a first nut is fitted on the right-hand thread section, and a second nut is fitted on the left-hand thread section; and
the first nut and the second nut are prevented by a guide from rotating with the right-hand and left-hand thread sections; and the other end of the first ligament is connected to the first nut and the other end of the second ligament is connected to the second nut.

4. A mechanical joint system according to claim 3, wherein the locations of the first connection of the first segment and the second connection of the second segment are adjustable.

5. A mechanical joint system according to claim 3, wherein each of the first nut and the second nut is replaced with a block with teeth engaged with and slidable along the right-hand and left-hand threaded sections, respectively, and the blocks are restrained by the guide.

6. A mechanical joint system according to claim 3, wherein the threaded cylinder have a second pair of a right-hand thread sections and a left-hand thread section, the second pair of the right-hand and left-hand thread sections is engaged with another pair of a first nut and a second nut, which are connected to a first segment and a second segment of another joint unit with ligaments.

7. A mechanical joint system according to claim 3, wherein at least one of the right-hand and left-hand thread sections has female threads, and the first nut and the second nut have threads matchable with the threads of the right-hand and left-hand threaded cylinders.

8. A mechanical joint system according to claim 3, wherein at least one thread section has a thread type different from the others.

9. A mechanical joint system according to claim 6, wherein at least one thread section has a thread type different from the others.

10. A mechanical joint system according to claim 3, wherein the first segment has a swivel axle (101) coaxial with the jointing portion, and the first and second ligaments are replaced with a rigid ligament comprising a gear which is also a portion of the swivel axle (101) of the first segment, a first rack extending from the first nut and a second rack extending from the second nut; the first and second racks being engaged with the gear; and when the first nut and the second nut are translated along the right-hand and left-hand thread sections respectively, the first rack and the second rack drive the gear to swivel the first segment relative to the second segment.

11. A mechanical joint system, comprising:
a joint unit including a first segment, a second segment, a jointing portion (A1) connecting the first segment and the second segment and allowing the first segment and the second segment to swivel about the jointing portion (A1), an extendible and retractable cylinder mechanism, a first ligament with one end connected to a first connection (b10) of the first segment and a second ligament with one end connected to a second connection (b20) of the first segment; wherein:
the extendible and retractable cylinder mechanism comprises a rail, a first cylinder, a second cylinder, a first block fitted to the first cylinder, a second block fitted to the second cylinder, and a driving device; and the first and second cylinders slide along the rail in opposite directions when the driving device is initiated to drive the first and second cylinders;
the first block and the second block are prevented from rotating with the first and second cylinders; and the other end of the first ligament is connected to the first block and the other end of the second ligament is connected to the second block.

12. A mechanical joint system according to claim 11, wherein the rail can be replaced with a shaft or a chamber able to guide the first and second cylinders to slide in opposite directions.

13. A mechanical joint system according to claim 11, wherein first cylinder and the second cylinder of the extendible and retractable cylinder mechanism are threaded, and the first block and the second block are threaded to engage with and movable along the first and second cylinders respectively.

14. A mechanical joint system according to claim 11, wherein the driving device is powered by hydraulic, pneumatic or magnetic force.

15. A mechanical joint system according to claim 13, wherein the driving device is powered by hydraulic, pneumatic or magnetic force.

16. A mechanical joint system according to claim 11, the locations of the first connection of the first segment and the second connection of the second segment are adjustable.

17. A mechanical joint system according to claim 13, the locations of the first connection of the first segment and the second connection of the second segment are adjustable.

18. A mechanical joint system according to claim 13, wherein the thread types of the pair of the first cylinder and the first block and the pair of the second cylinder and the second block are different.

19. A mechanical joint system according to claim 13, wherein the pair of the first cylinder and the first block is threaded with a right-hand thread and the pair of the second cylinder and the second block is threaded with a left-hand thread.

20. A mechanical joint system according to claim 11, wherein the first and second ligaments are replaced with a rigid ligament comprising a gear swivelable with the first segment about the jointing portion (A1), a first rack extending from the first block and a second rack extending from the second block; the first and second racks being engaged with the gear; and when the first block and the second block are relatively moved, the first rack and the second rack drive the gear to swivel the first segment relative to the second segment.

21. A mechanical joint system according to claim 13, wherein the first and second ligaments are replaced with a rigid ligament comprising a gear swivelable with the first segment about the jointing portion (A1), a first rack extending from the first block and a second rack extending from the second block; the first and second racks being engaged with the gear; and when the first block and the second block are relatively moved, the first rack and the second rack drive the gear to swivel the first segment relative to the second segment.

22. A mechanical joint system, comprising:
a joint unit including a first segment, a second segment, a jointing portion (A1) connecting the first segment and the second segment and allowing the first segment and the second segment to swivel about the jointing portion (A1), a threaded cylinder, a nut movable along the threaded cylinder and a force transmitting device; wherein:
the first segment has a swivel axle (101) coaxial with the jointing portion (A1);
the threaded cylinder has at least a right-hand thread section or a left-hand thread section for the nut to be fitted on, and the nut is prevented by a guide from rotating with the threaded cylinder; and
the force transmitting device comprises a gear which is also a portion of the swivel axle (101) of the first segment, and a rack extending from the nut and being engaged with the gear; and when the nut is moved along the threaded cylinder, the rack drives the gear to swivel the first segment relative to the second segment.
